(19) United States
(12) Patent Application Publication
(10) Pub. No.: US 2023/0307107 A1
(43) Pub. Date: Sep. 28, 2023

(54) PERSONALIZED TREATMENT USING METABOLIC FINGERPRINT BASED SIMULATION

(71) Applicant: CITY OF HOPE, Duarte, CA (US)

(72) Inventors: Fouad KANDEEL, Duarte, CA (US); Christopher ORR, Duarte, CA (US)

(73) Assignee: CITY OF HOPE, Duarte, CA (US)

(21) Appl. No.: 18/000,636

(22) PCT Filed: Jun. 4, 2021
(86) PCT No.: PCT/US2021/035987

§ 371 Date: Dec. 2, 2022

Related U.S. Application Data
(60) Provisional application No. 63/035,258, filed on Jun. 5, 2020.

(51) Int. Cl. G16H 20/10; G16H 10/60; G16H 20/30; G16H 20/60; G16H 50/50

(52) U.S. Cl. CPC G16H 20/10; G16H 10/60; G16H 20/30; G16H 20/60; G16H 50/50

(57) ABSTRACT

Providing a personalized treatment by using a metabolic fingerprint is described. In one aspect, health information regarding a patient can be received and used to generate a metabolic fingerprint of that patient and using the metabolic fingerprint, characteristics of the glucose of the patient can be determined and used to generate a drug therapy plan to aid in the regulation of glucose within the patient. Additionally, using a patient's characteristics, a safe cardiopulmonary fitness of a patient can be determined.

### PRIORITY CLAIM

This application claims the benefit of U.S. Provisional Patent Application No. 63/035,258, filed Jun. 5, 2020, the contents of which is hereby incorporated by reference in its entirety, including drawings.

### TECHNICAL FIELD

This disclosure relates to personalized treatments, and in particular providing a personalized treatment by using a metabolic fingerprint.

### BACKGROUND

Diabetes mellitus is one of the most serious global health threats of the modern era. In 2010, diabetes mellitus affected 285 million people worldwide. If current trends continue, 438 million people will have diabetes by 2030. As a well-established risk factor for heart disease, stroke, and kidney failure, diabetes-related deaths will also double between 2005 and 2030. With as many as half of all diabetic patients remaining undiagnosed, these already sizable figures are likely gross underestimations. Longer term projections are even worse, for example, the Center for Disease Control (CDC) has recently estimated that 76 million Americans (one out of every three peoples) will have diabetes by 2050, and these estimates will hold-up even if obesity rates remain static. Such high estimates of future disease incidence suggest that cost of care for diabetes will overwhelm if not bankrupt the American health care system. The outlook is even worse in many other countries. More than 50% of the world's expenditure on diabetes care occurs in the United States, and the anticipated rates of increase in disease incidence are much less than in lower-income countries, where 70% of diabetics currently reside. These lower-income countries also have inadequate health care systems. Moreover, 46% of diabetics worldwide are of working age (40-59 years) and the disease is increasingly occurring at an earlier age, suggesting that the cost of treating diabetes and its complications over the life span of affected individuals together with the loss in their productivity will overwhelm the world economy.

Attempts to improve long-term therapeutic outcomes for patients have been largely ineffective due to the inconsistent implementation of standards of care for diabetes, inefficient healthcare delivery systems, inadequate numbers of trained physicians, and a general lack of financial resources in many parts of the world, and are frequently confounded by a lack of patient compliance and reliable long-term outcome data, particularly within and among countries. Thus, there is a critical need for a cost-effective solution to simplify diabetes management, reduce physician time/effort spent on expensive and unstructured treatment plans, and simultaneously improve patient acceptance of behavioral modification and therapeutic drug regimens that can be applied on a global scale.

### BRIEF DESCRIPTION OF THE DRAWINGS

FIG. 1 illustrates an example of generating a personalized drug therapy for a patient.

FIG. 2 illustrates an example of a block diagram for generating a physical activity program based on patient cardiopulmonary fitness, patient preferences, and patient physical limitations.

FIG. 3 illustrates an example of a block diagram for providing alternative meal options to adjust meal content of carbohydrates, saturated fats, meal size, and fulfillment of nutritional density.

FIG. 4 illustrates an example of a device for generating a personalized drug therapy for a patient.

### DETAILED DESCRIPTION

This disclosure describes devices and techniques for generating a personalized treatment for a patient using a simulation based on a metabolic fingerprint. In one example, a patient can use a computing device such as a mobile device (e.g., laptop, smartphone, tablet) or a desktop computer to provide information regarding the patient's demographics (e.g., the patient's age, height, weight), dietary information (e.g., meals that the patient eats), exercise information (e.g., how often and how long the patient exercises and what physical activities the patient performs when the patient exercises, such as running on a treadmill), and drug information (e.g., the drugs that the patient currently takes). This information can represent a profile of the patient that can be provided to a server. That server can then generate a metabolic fingerprint of the patient based on the information provided via their profile. The metabolic fingerprint can be used to determine an estimate of the patient's glucose levels or an estimate of the generation of glucose within the patient's body. Using the metabolic fingerprint and the patient's information, a simulation can then be performed to generate a personalized drug therapy for the patient. For example, information regarding the dosage of a drug (e.g., an initial dosage, an increased dosage of a drug that the patient is currently taking, a decreased dosage of the drug that the patient is currently taking) can be determined and provided to a healthcare provider to guide the patient on a new drug therapy regimen.

In another example, the dietary information indicated in the patient information can be analyzed to determine the constituent parts of the meals that the patient eats. For example, if the patient eats spaghetti, then the amounts of fiber, carbohydrates, saturated fats, and other nutrients that constitute the meal can be determined by accessing a database providing such information. Those constituent parts of the meal can then be compared with other constituent parts of other meals. The other meals that are similar (based on the corresponding constituent parts) to the meal that the patient prefers to eat can then be identified and recommended to the patient to eat. The similarities between the meal that the patient eats and the other meals can be identified using a root mean square (RMS) error statistical analysis. A similar procedure can also be used to provide changes to the patient's exercise routine. Moreover, changes to the patient's diet and/or exercise can be considered first before recommending a new drug therapy regimen.

Implementation of these examples can result in a derivation of an individualized treatment for patients that can be quickly and easily provided and, therefore, reducing the likelihood of developing diseases such as diabetes or reducing the effects of such diseases. The treatment can include alterations to the meals that the patient consumes, exercise that the patient engages in, or drug therapy that the user follows.

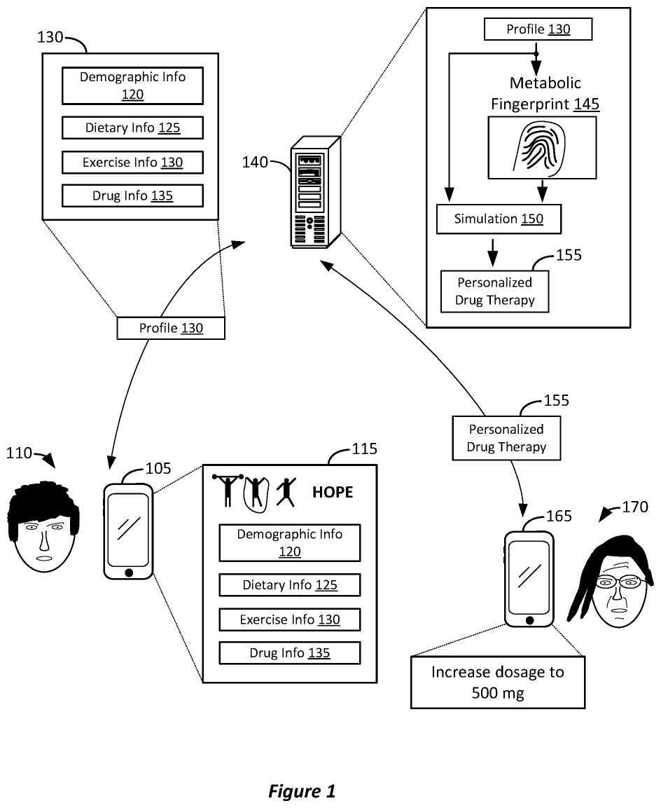

In more detail, FIG. 1 illustrates an example of generating a personalized drug therapy for a patient. In FIG. 1, patient 110 can use a computing device 105 (e.g., a smartphone, tablet, desktop computer, laptop computer, smart watch) to use an application 115 to aid in the management of the patient's health, for example, management of diabetes. Using application 115, patient 110 can provide demographic information 120 regarding the patient such as the patient's age, weight, height, body mass index (BMI), ethnicity, race, income, or other characteristics of the patient useful for statistical characterization to identify segments, or portions, of the population that patient 110 might be identified with. Demographic information 120 can be input in via text (e.g., by typing in a text box), selected (e.g., patient 110 can select radio buttons, drop down boxes, etc. in a graphical user interface (GUI)), or imported (e.g., from a social network profile that patient 110 has set up).

Other types of health information including dietary information 125, exercise information 130, and drug information 135 can be provided in a similar manner. Dietary information 125 can indicate the meals that patient 110 prefers to eat or has recently eaten. For example, dietary information 125 can indicate that patient 110 has eaten meals such as spaghetti, broccoli cheddar soup, and cheese enchiladas.

Exercise information 130 can indicate the types of exercises or physical activity that patient 110 prefers to perform or has recently performed. For example, exercise information 130 can indicate that patient 110 has jogged five miles every other day for the past week. In some implementations, exercise information 130 can also include health-related data from a wearable device such as an activity tracker (or fitness tracker), smart watch, etc. that determines and stores (in memory) the patient's heart rate, duration of exercise, or other information related to the exercises or physical activity that the patient has performed.

Drug information 135 can indicate the drugs or types of drugs that patient 110 is currently taking or has taken in the past (e.g., the patient's drug intake history). For example, drug information 135 can indicate that patient 110 is currently taking 200 milligrams (mg) of Metformin, a drug used for the treatment of type 2 diabetes.

This information provided via application 115 can be provided as profile 130 in FIG. 1 to server 140. Server 140 can analyze the information provided, and perform additional functionalities as discussed later herein, to generate a personalized treatment plan for patient 110, for example, recommending different meals to eat (based on dietary information 120 and demographic information 120), different exercises to perform (based on exercise information 130 and demographic information 120), and changes to a drug therapy or regimen (based on drug information 135 and demographic information 120). Some of these recommendations can be provided back to patient 110. For example, alternative meal options for patient 110 to consider can be determined by server 140 and data indicating the alternative meal options can be provided back to computing device 105 to be displayed in a new GUI of application 115. Other recommendations can be provided to computing device 165 of doctor 170. That is, the doctor of patient 110 can receive some of the recommendations as well. For example, doctor 170 can receive via computing device 165 information regarding changes to the patient's drug therapy. The doctor can then provide a new prescription for patient 110.

In this example, computing device 105 communicates with server 140 and server 140 uses the information provided in profile 130 to generate the recommendations. However, in other implementations, the functionality and features described with respect to server 140 can be implemented within application 115 of computing device 105. That is, application 115 can also analyze demographic information 120, dietary information 125, exercise information 130, drug information 135 and generate the recommendations discussed herein. In some implementations, the various databases discussed herein can be accessed by computing device 105 and the functionality, calculations, etc. described herein can be performed by computing device 105.

In FIG. 1, server 140 receives profile 130 and can generate metabolic fingerprint 145 based on demographic information 120, dietary information 125, exercise information 130, and drug information 135. For example, metabolic fingerprint 145 can indicate the patient's glucose profile (or glycemic status) providing some indication regarding glucose levels or glucose production within the patient's body. Management of glucose levels of patients with diabetes or at the risk of developing diabetes due to impairment of production of insulin can be an important part of a patient's medical treatment.

In some implementations, metabolic fingerprint 145 can be a mathematical model used in a simulation (of the patient's glucose profile) to provide recommendations to changes in the patient's diet, exercise, or drug therapy. In some implementations, metabolic fingerprint 145 can be determined via a consideration of certain mathematical equations or algorithmically. For example, metabolic fingerprint 145 can be generated based on a consideration of the information provided via profile 130.

Server 140 can then run simulation 150 using metabolic information 145 and the information of profile 130 (e.g., demographic information 120, dietary information 125, exercise information 130, and drug information 135) to generate a recommendation regarding the diet, exercise, or drug therapy of patient 110. For example, in FIG. 1, personalized drug therapy 155 can be generated by running simulation 150 to determine how the patient's glucose characteristics such as its variability of glucose levels changes based on the patient's demographics, diet, exercise, and current drug therapy. Based on those determined changes, personalized drug therapy 155 can be generated as a recommendation. For example, personalized drug therapy 155 can be generated indicating that patient 110 should increase a dosage of a drug that the patient is currently taking if it is determined via simulation 150 that the patient's glucose levels would increase based on the current meals, exercise, and drug therapy for people with the patient's demographics. This can be determined by having simulation 150 use metabolic fingerprint 145 and profile 140. In FIG. 1, this results in doctor 170 receiving personalized drug therapy 155 as a recommendation for the patient's prescription to increase to 500 mg to help aid in the control of glucose.

In some implementations, metabolic fingerprint 145 can be provided by another server. For example, server 140 can provide profile 130 to another server to generate metabolic fingerprint 145. Metabolic fingerprint 145 can then be provided to server 140 to use for simulation 150.

Thus, patient 110 can be provided with a personalized treatment plan that can be quickly and easily provided and, therefore, reduce the likelihood of developing diabetes or reduce (or control) the effects of diabetes. Additionally, because the personalized treatment is generated using sophisticated techniques (including using a metabolic fingerprint), the personalized treatment plan can be more objective and influenced by fewer subjective factors between the patient and the doctor.

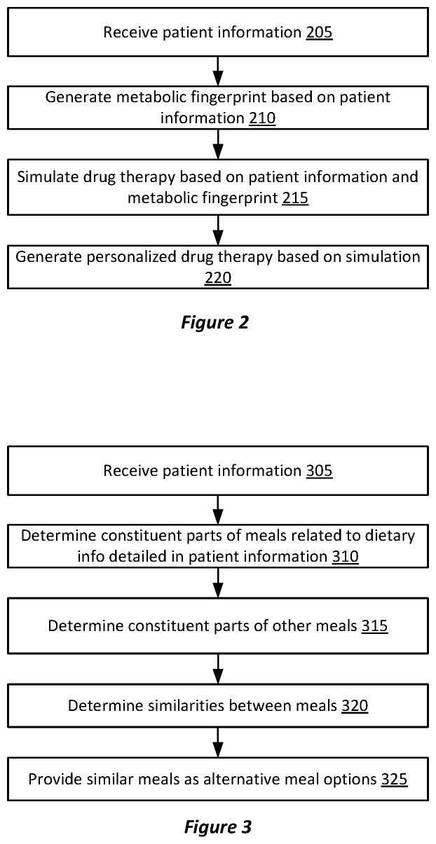

FIG. 2 illustrates an example of a block diagram for generating a personalized drug therapy for a patient. In FIG. 2, patient information can be received (205) to aid in the management of diseases such as diabetes. For example, in FIG. 1, profile 130 indicating the patient's demographic information 120, diet information 125, exercise information 130, and drug information 135 can be received by server 140. Using profile 130, a metabolic fingerprint based on the patient's information can be generated (210). For example, in FIG. 1, metabolic fingerprint 145 can be generated to provide a model for the patient's glucose variability. In some implementations, the foods indicated in dietary information 125 can be “broken down” into their constituent parts to determine the nutritional content of the foods, as discussed later below. These constituent parts can, in some implementations, be used to generate metabolic fingerprint 145.

This model can be used along with the patient information to simulate a drug therapy (215). For example, in FIG. 1, personalized drug therapy 155 can be generated recommending a change in the patient's drugs or drug dosage. This recommendation can be made by running a simulation that determines how the patient information provided changes the patient's glucose variability as defined by metabolic fingerprint 145. For example, different meals, exercises, demographics, and drugs can result in different changes in the glucose levels of the same patient. Thus, different information can result in different effects regarding the glucose levels of the patient. Based on the changes, a personalized drug therapy based on the simulation can be provided (220). For example, in FIG. 1, personalized drug therapy 155 can be generated to include a recommendation to increase the dosage of an existing drug that patient 110 is taking (as indicated in drug information 135) such that the patient's glucose variability is constrained.

In some implementations, a change to a patient's diet or exercise can be recommended first before providing a change to the patient's drug therapy. This might be done because changes to diet or exercise might be preferred rather than engaging with medication. In some implementations, this can be performed on a patient's electronic device, for example, a desktop computer, smartphone, etc. or a combination of the patient's electronic device and a server. FIG. 3 illustrates an example of a block diagram for providing alternative meal options. In FIG. 3, patient information can be received. For example, as previously discussed, profile 130 indicating demographic information 120, dietary information 125, exercise information 130, and drug information 135 can be provided.

If dietary information 125 indicates meals that patient 110 in FIG. 1 eats, then the constituent parts of those meals can be determined (310). For example, if patient 110 indicates that spaghetti is eaten, then server 140 can determine the nutrients that spaghetti is made of, for example, by accessing a food information database (either part of server 140 or accessible to server 140) storing such information such as a United States Department of Agriculture (USDA) food composition database providing details such as the nutrients of different meals or food products. In one example, the constituent parts can indicate the amounts of fiber, carbohydrates, saturated fat, trans fat, cholesterol, protein, sugars, vitamins, etc.

Constituent parts of other meals can then be determined (315). For example, other meals within the food information database can be identified as possible alternatives for the patient to choose to eat to maintain their glucose variability within an acceptable range to prevent or manage diabetes. The constituent parts of those meals can be identified using the food information database. Once the constituent parts of the patient's meals and the other meals found using the food information database are identified, similarities between these meals can be determined (320). In some implementations, the patient can use a camera (e.g., on a smartphone) and take photos of food consumed over a time period (e.g., seven days). Various algorithms can be used to determine the constituent parts of those foods and used to determine positive and negative constituent parts representing good nutrients and bad nutrients for the patient's health, respectively.

Similarities between meals can be determined using a root mean square (RMS) statistical analysis as an indication for a measure of error, or differences between meals. For example, if a patient's meal as indicated in dietary information 125 indicates that the user eats spaghetti, then the constituent parts of spaghetti can include 3 grams (g) of fiber. Server 140 can determine that another meal such as quinoa includes 3.1 g of fiber. The RMS analysis can consider the differences between the fiber content (0.1 g of fiber in the example) as well as other pairs of other constituents (e.g., the differences between the amount of carbohydrates between the patient's meal and the alternative meal). The RMS calculated for each constituent of an alternative meal can then be added up and compared with the added RMS for the constituents of another meal. A meal with the lowest added RMS (i.e., its constituent parts represent a meal with close nutritional similarity to the meal indicated by the patient) can be the meal with the closest nutritional similarity and, therefore, provided as an alternative for the patient to consider (325). Other meals can also be provided, for example, server 140 can provide data to be displayed in a GUI of application 115 with a list of meals in a descending order of less similarity (i.e., the most similar meal can be listed at the top of the list).

This allows a patient to restructure their daily meal intake to be more conducive to diabetes management by avoiding some constituent parts that can worsen diabetes or lead to diabetes. For example, the carbohydrates intake of the patient can be reduced over a time period (e.g., twenty-four hours), the percentage of daily calorie intake provided by a meal can be adjusted (e.g., reduced), the amount of saturated fats eaten can be reduced, etc.

To provide changes to a patient's exercise routine, the patient's baseline energy can be determined using the information provided in profile 130. For example, exercise information 130 can provide information regarding the patient's physical activities such as how often and how long (e.g., length and duration) they exercise, such as whether they walk to work and how long that walk is, etc. Server 140 can receive this information and provide alternative exercises that the patient can perform while an energy expenditure corresponding to the baseline energy of the patient. In some implementations, computing device 105 can determine and provide the alternative exercises (e.g., without providing information to server 140). For example, the suggested alternative exercises can use the same baseline energy to perform as the baseline energy that the patient expends when performing the exercises indicated in exercise information 130.

In some implementations, the patient can manage their diabetes by having their cardiopulmonary fitness analyzed and/or determined. For example, computing device 105 can be provided information (e.g., input by patient 110 using a GUI of application 115) including various characteristics and information regarding the patient and their activities, for example, the demographics (e.g., age, gender, etc.) of the patient, current medical information regarding the patient (e.g., weight, resting heart rate, etc.), and exercise information (e.g., physical activities performed by patient 110 such as how much they walk, run, flights of stairs climbed, etc.). Computing device 105 can receive this information and determine a cardiopulmonary fitness of patient 110 based on the demographics of the patient and the patient's resting heart rate. This can provide a model of the patient's health. Using that cardiopulmonary fitness, changes or adjustments can be made to the physical activities performed by the patient as indicated in the exercise information. For example, additional or different exercises or activities can be recommended, the same exercises or activities at different durations (e.g., longer) can be recommended, etc.

Later, when patient 110 follows the recommendations of additional or different exercises, the cardiopulmonary fitness of that patient can be determined again based on the patient's new exercise information. A change in the cardiopulmonary fitness can then be determined. If the change is positive (e.g., the patient's health has improved), then this can be provided to patient 110 via a message, for example, generated on a GUI of application 115. If the change is negative (e.g., the patient's cardiopulmonary fitness has not improved or decreased to a lower level of fitness) then patient 110 can be informed and new recommendations can be provided.

In some implementations, changes to diet and exercise can be determined first. If the changes cannot occur, then drug therapy can be recommended. For example, if alternative meals that can constrain the patient's glucose levels cannot be recommended because there are no available meals similar to what the patient provided in dietary information 125, then server 140 can recommend a personalized drug therapy by performing simulation 150 in FIG. 1 as described in FIGS. 1 and 2. Likewise, if no changes to the exercise of the patient can result in the patient's glucose level being within an acceptable range, then a personalized drug therapy can be provided. In some implementations, changes to diet, exercise, and drug therapy can be provided in a message to doctor 170 in FIG. 1. For example, in FIG. 1, personalized drug therapy 155 can be provided as a message to doctor 170 along with the information regarding diet and exercise (e.g., changes to diet and exercise, as previously discussed). Thus, improvements to a patient's physical activity using the patient's cardiopulmonary fitness as a guide, adjustments to food intake (e.g., nutritional content) by suppressing bad elements and favoring good elements, and modifying the patient's medical care (e.g., drug therapy) can be performed.

In some implementations, patient 110 in FIG. 1 can be provided messages on a GUI of application 115 indicating how they are conforming to any changes to their exercise, diet, and/or drug therapy. For example, if the patient is performing well (e.g., sticking to the recommended changes), then they can be provided a message so and, therefore, be encouraged to continue the recommended regimen. By contrast, if the patient is not performing well (e.g., deviating from the recommended changes), then they can be provided a message to encourage them to get back to the regimen.

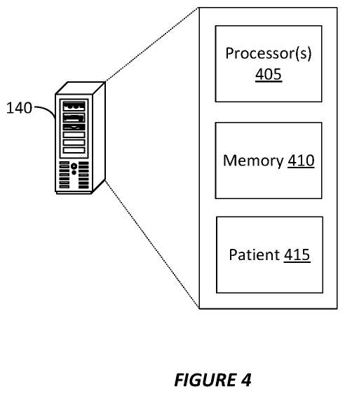

FIG. 4 illustrates an example of a device for generating a personalized drug therapy for a patient. In FIG. 4, server 140 includes a processor 405, memory 410, as well as other types of hardware such as non-volatile memory, an interface device, camera, microphones, speakers, etc. to implement patient logic 630 providing the techniques disclosed herein. In other implementations, computing devices 105 and 165 also include similar components to implement the techniques disclosed herein. Various common components (e.g., cache memory) are omitted for illustrative simplicity. The server is intended to illustrate a hardware device on which any of the components described in the example of FIGS. 1-3 (and any other components described in this specification) can be implemented. The components of the server can be coupled together via a bus or through some other known or convenient device.

The processor 405 may be, for example, a microprocessor circuit such as an Intel Pentium microprocessor or Motorola power PC microprocessor. One of skill in the relevant art will recognize that the terms “machine-readable (storage) medium” or “computer-readable (storage) medium” include any type of device that is accessible by the processor. Processor 605 can also be circuitry such as an application specific integrated circuits (ASICs), complex programmable logic devices (CPLDs), field programmable gate arrays (FPGAs), structured ASICs, etc.

The memory is coupled to the processor by, for example, a bus. The memory can include, by way of example but not limitation, random access memory (RAM), such as dynamic RAM (DRAM) and static RAM (SRAM). The memory can be local, remote, or distributed.

The bus also couples the processor to the non-volatile memory and drive unit. The non-volatile memory is often a magnetic floppy or hard disk; a magnetic-optical disk; an optical disk; a read-only memory (ROM) such as a CD-ROM, EPROM, or EEPROM; a magnetic or optical card; or another form of storage for large amounts of data. Some of this data is often written, by a direct memory access process, into memory during the execution of software in the computer. The non-volatile storage can be local, remote or distributed. The non-volatile memory is optional because systems can be created with all applicable data available in memory. A typical computer system will usually include at least a processor, memory, and a device (e.g., a bus) coupling the memory to the processor.

The software can be stored in the non-volatile memory and/or the drive unit. Indeed, storing an entire large program in memory may not even be possible. Nevertheless, it should be understood that for software to run, it may be necessary to move the software to a computer-readable location appropriate for processing, and, for illustrative purposes, that location is referred to as memory in this application. Even when software is moved to memory for execution, the processor will typically make use of hardware registers to store values associated with the software and make use of a local cache that, ideally, serves to accelerate execution. As used herein, a software program is can be stored at any known or convenient location (from non-volatile storage to hardware registers).

The bus also couples the processor to the network interface device. The interface can include one or more of a modem or network interface. Those skilled in the art will appreciate that a modem or network interface can be considered to be part of the computer system. The interface can include an analog modem, an ISDN modem, a cable modem, a token ring interface, a satellite transmission interface (e.g., “direct PC”), or other interface for coupling a computer system to other computer systems. The interface can include one or more input and/or output devices. The input and/or output devices can include, by way of example but not limitation, a keyboard, a mouse or other pointing device, disk drives, printers, a scanner, and other input and/or output devices, including a display device. The display device can include, by way of example but not limitation, a cathode ray tube (CRT), a liquid crystal display (LCD), or some other applicable known or convenient display device.

In operation, the server can be controlled by operating system software that includes a file management system, such as a disk operating system. The file management system is typically stored in the non-volatile memory and/or drive unit and causes the processor to execute the various acts required by the operating system to input and output data, and to store data in the memory, including storing files on the non-volatile memory and/or drive unit.

Some items of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electronic or magnetic signals capable of being stored, transferred, combined, compared, and/or otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, those skilled in the art will appreciate that throughout the description, discussions utilizing terms such as “processing” or “computing” or “calculating” or “determining” or “displaying” or “generating” or the like refer to the action and processes of a computer system or similar electronic computing device that manipulates and transforms data represented as physical (electronic) quantities within the computer system's registers and memories into other data similarly represented as physical quantities within the computer system's memories or registers or other such information storage, transmission, or display devices.

The algorithms and displays presented herein are not inherently related to any particular computer or other apparatus. Various general-purpose systems may be used with programs in accordance with the teachings herein, or it may prove convenient to construct more specialized apparatuses to perform the methods of some embodiments. The required structure for a variety of these systems will be apparent from the description below. In addition, the techniques are not described with reference to any particular programming language, and various embodiments may thus be implemented using a variety of programming languages.

In further embodiments, the server operates as a standalone device or may be connected (e.g., networked) to other machines. In a networked deployment, the server may operate in the capacity of a server or of a client machine in a client-server network environment or may operate as a peer machine in a peer-to-peer (or distributed) network environment.

In some embodiments, the server includes a machine-readable medium. While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database and/or associated caches and servers) that store the one or more sets of instructions. The term “machine-readable medium” and “machine-readable storage medium” should also be taken to include any medium that is capable of storing, encoding, or carrying a set of instructions for execution by the machine, and which causes the machine to perform any one or more of the methodologies or modules of the presently disclosed technique and innovation.

In general, the routines executed to implement the embodiments of the disclosure may be implemented as part of an operating system or a specific application, component, program, object, module, or sequence of instructions referred to as “computer programs.” The computer programs typically comprise one or more instructions set at various times in various memory and storage devices in a computer that, when read and executed by one or more processing units or processors in a computer, cause the computer to perform operations to execute elements involving various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally, regardless of the particular type of machine- or computer-readable media used to actually effect the distribution.

Further examples of machine-readable storage media, machine-readable media, or computer-readable (storage) media include, but are not limited to, recordable type media such as volatile and non-volatile memory devices, floppy and other removable disks, hard disk drives, optical disks (e.g., Compact Disc Read-Only Memory (CD-ROMS), Digital Versatile Discs, (DVDs), etc.), among others, and transmission type media such as digital and analog communication links.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice-versa. The foregoing is not intended to be an exhaustive list in which a change in state for a binary one to a binary zero or vice-versa in a memory device may comprise a transformation, such as a physical transformation. Rather, the foregoing is intended as illustrative examples.

A storage medium may typically be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe certain principles and practical applications, thereby enabling others skilled in the relevant art to understand the subject matter, the various embodiments and the various modifications that are suited to the particular uses contemplated.

While embodiments have been described in the context of fully functioning computers and computer systems, those skilled in the art will appreciate that the various embodiments are capable of being distributed as a program product in a variety of forms and that the disclosure applies equally regardless of the particular type of machine- or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details while still being encompassed by the specification. As noted above, particular terminology used when describing certain features or aspects of various embodiments should not be taken to imply that the terminology is being redefined herein to be restricted to any specific characteristics, features, or aspects of the disclosed technique with which that terminology is associated. In general, the terms used in the following claims should not be construed to limit the disclosure to the specific embodiments disclosed in the specification, unless those terms are explicitly defined herein. Accordingly, the actual scope of the technique encompasses not only the disclosed embodiments but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the technique be limited not by this Detailed Description, but rather by any claims that issue on an application based hereon. Accordingly, the disclosure of various embodiments is intended to be illustrative, but not limiting, of the scope of the embodiments, which is set forth in the following claims.

From the foregoing, it will be appreciated that specific embodiments of the invention have been described herein for purposes of illustration, but that various modifications may be made without deviating from the scope of the invention. Accordingly, the invention is not limited except as by the appended claims.

We claim:

1. A method for using a metabolic fingerprint to simulate glucose levels of a patient, the method comprising:
receiving, by a server, information regarding the patient, the information indicating demographics of the patient, dietary information representing meals that the patient eats, exercise information representing physical activities performed by the patient, and drug information representing a drug therapy taken by the patient;
generating, by the server, a metabolic fingerprint of the patient based on the demographics of the patient, the dietary information, exercise information, and drug information, the metabolic fingerprint representing a glucose profile of the patient;
simulating, by the server, characteristics of glucose of the patient using the metabolic fingerprint and the information regarding the patient;
determining, by the server, that the characteristics of the glucose of the patient include a glucose level that cannot be constrained within a threshold range by an available change in the physical activities performed by the patient or an available change in the meals that the patient eats;
determining, by the server, a drug therapy providing a dosage of a medication to control the glucose level of the patient within the threshold range based on the determination that the characteristics of the glucose of the patient include the glucose level that cannot be constrained within a threshold range by an available change in the physical activities performed by the patient or an available change in the meals that the patient eats; and
providing, by the server, an indication to a computing device regarding the drug therapy to provide the patient with the dosage of the medication to control the glucose level.

2. An electronic device, comprising:
one or more processors; and
memory storing instructions, wherein the processor is configured to execute the instructions such that the processor and memory are configured to:
receive health information regarding a patient;
generate a metabolic fingerprint representing a glucose profile of the patient based on the health information regarding the patient;
determine characteristics of glucose of the patient based on the metabolic fingerprint and the health information; and
generate a drug therapy plan to regulate the glucose of the patient based on the characteristics of the glucose of the patient.

3. A computer program product, comprising one or more non-transitory computer-readable media having computer program instructions stored therein, the computer program instructions being configured such that, when executed by one or more computing devices, the computer program instructions cause the one or more computing devices to:
receive information regarding a patient;
generate a metabolic fingerprint representing a glucose profile of the patient based on the health information regarding the patient;
simulate characteristics of glucose of the patient based on the metabolic fingerprint and the information regarding the patient; and
determine a dosage of a medication used to regulate the glucose of the patient based on the characteristics of the glucose of the patient.

4. A method for assisting a patient in managing diabetes, the method comprising:
receiving, by a processor, information regarding the patient, the information indicating demographics of the patient, a resting heart rate of the patient, and exercise information representing physical activities performed by the patient;
determining, by the processor, a cardiopulmonary fitness of the patient based on the demographics of the patient and the resting heart rate of the patient;
providing, by the processor, recommendations to adjustments to the physical activities performed by the patient based on the cardiopulmonary fitness and the physical activities performed by the patient to manage the diabetes of the patient;
determining a change of the cardiopulmonary fitness of the patient based on the recommendations to adjustments to the physical activities; and
informing the patient regarding the change of the cardiopulmonary fitness.

5. A method for assisting a patient in managing diabetes, the method comprising:
receiving, by a processor, information regarding the patient, the information indicating demographics of the patient, and dietary information representing meals that the patient eats;
determining, by the processor, constituent elements making up the meals that the patient eats;
determining, by the processor, a first set of constituent elements corresponding to positive dietary nutrients, and a second set of constituent elements corresponding to negative dietary nutrients; and
recommending adjustments to the first set of constituent elements and the second set of constituent elements to reduce consumption of the negative dietary nutrients and increase consumption of the positive dietary nutrients to manage the diabetes of the patient.
